(19) United States
(12) Patent Application Publication
(10) Pub. No.: US 2010/0183501 A1
(43) Pub. Date: Jul. 22, 2010

(54) Medical Devices With Nanotextured Titanium Coating

(75) Inventor: Iskender Matt Bilge, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/355,583

(22) Filed: Jan. 16, 2009

(51) Int. Cl. A61F 2/00; A61K 38/00; A61K 31/7088

(52) U.S. Cl. 424/1.11; 424/423; 514/2; 977/773; 977/734; 514/44 R

(57) ABSTRACT

A system for treating a vascular condition includes a catheter, a stent disposed on the catheter, the stent having a stent framework including a core, the core having an outer surface and a pure titanium layer deposited on the outer surface of the core and a nanotextured surface formed in the deposited pure titanium layer. A method of manufacturing a stent for treating a vascular condition includes depositing a uniformly dense and uniformly thick layer of pure titanium onto an outer surface of a stent framework core, placing stent framework into a running chamber having a cathode and electrolyte solution, connecting stent framework and cathode to a power source operably connected to the running chamber, applying a power source to the attached stent framework and cathode for a predetermined length of time and at a predetermined voltage and forming a nanotextured surface within the deposited titanium layer.

### TECHNICAL FIELD

This invention relates generally to biomedical devices. More specifically, the invention relates to implantable stents and other medical devices having a nanotextured titanium coating.

### BACKGROUND OF THE INVENTION

Implantable devices often include a therapeutic agent as part of a therapeutic agent coating or as part of the material forming the implantable device. With these devices, the therapeutic agent contained in the coating begins to elute or diffuse from the device surface soon after implantation resulting in a burst of therapeutic agent at the treatment site. In some situations an initial burst of therapeutic agent may be useful. However, in other situations a sustained, controlled release of the therapeutic agent from the device surface is desired. Polymers, mixed with the therapeutic agent, have been used to control the release rate of the therapeutic agent from the device surface. However, there is some evidence that the use of polymers in drug coatings may not be suitable for some applications.

In an effort to reduce or eliminate the use of polymers in therapeutic coatings, some medical devices, including stents, include a porous surface. For some applications, a drug or other therapeutic agent is deposited in the pores for elution at a treatment site after implantation. In other devices, the pores may also increase biocompatibility by promoting cellular ingrowth. Several methods have been developed for manufacturing medical devices with large pores for drug deposition. However, these methods of manufacture are not suitable for providing a nanopores to a device structure. Methods of producing nanopores have been developed for large medical devices composed of a pure metal or having a sheet of pure metal on an outer surface. These methods have proven inadequate for use in smaller medical devices such as stents, especially stents having a composite structure composed of layers of material.

Pure titanium is often used for implantable devices such as hip replacements, dental and orthodontic implants. However, for some stent applications the use of pure titanium is often impractical or is not suitable for the particular application. Further, pure titanium may not provide performance characteristics that meet the needs of a particular application as would a composite material or an alloy or another metal such as stainless steel. Where titanium would be useful for stent applications, attempts to provide a stent with a pure titanium outer layer have been unsuccessful.

It would therefore be desirable, to provide an implantable medical device that would overcome the limitations and disadvantages described above.

### SUMMARY OF THE INVENTION

One aspect of the present invention provides a system for treating a vascular condition. The system includes a catheter, a stent disposed on the catheter, the stent having a stent framework including a core, the core having an outer surface and a pure titanium layer deposited on the outer surface of the core and a nanotextured surface formed in the deposited pure titanium layer.

Another aspect of the invention provides a stent for treating a vascular condition. The stent includes a stent framework including a core, the core having an outer surface, a pure titanium layer deposited on the outer surface of the core; and a nanotextured surface formed in the deposited pure titanium layer.

Another aspect of the invention provides a method of manufacturing a stent for treating a vascular condition. The method includes depositing a uniformly dense and uniformly thick layer of pure titanium onto an outer surface of a stent framework core, placing stent framework into a running chamber having a cathode and electrolyte solution, connecting stent framework and cathode to a power source operably connected to the running chamber, applying a power source to the attached stent framework and cathode for a predetermined length of time and at a predetermined voltage and forming a nanotextured surface within the deposited titanium layer

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of a system for treating a vascular condition comprising a therapeutic agent carrying stent coupled to a catheter, in accordance with one embodiment of the present invention;

FIG. 2 illustrate one embodiment of a stent framework of a stent suitable for use in the system illustrated in FIG. 1, in accordance with the present invention;

FIGS. 3A to 3D illustrate serial cross sections of another embodiment of a stent framework of a stent suitable for use in the system illustrated in FIG. 1, in accordance with the present invention; and

FIG. 4 is a flow diagram of a method for manufacturing a stent for treating a vascular condition, in accordance with the present invention.

### DETAILED DESCRIPTION

The invention will now be described by reference to the figures wherein like numbers refer to like elements. The terms “distal” and “proximal” are used herein with reference to the treating clinician during the use of the catheter system; “distal” indicates an apparatus portion distant from, or a direction away from the clinician and “proximal” indicates an apparatus portion near to, or a direction towards the clinician.

The present invention is directed to a system and method for preparing a medical device for treating abnormalities of the cardiovascular system. The below description is further directed to an implantable stent and system for treating abnormalities of the cardiovascular system. Those with ordinary skill in the art will appreciate that the below described invention can be applied to other implantable medical devices.

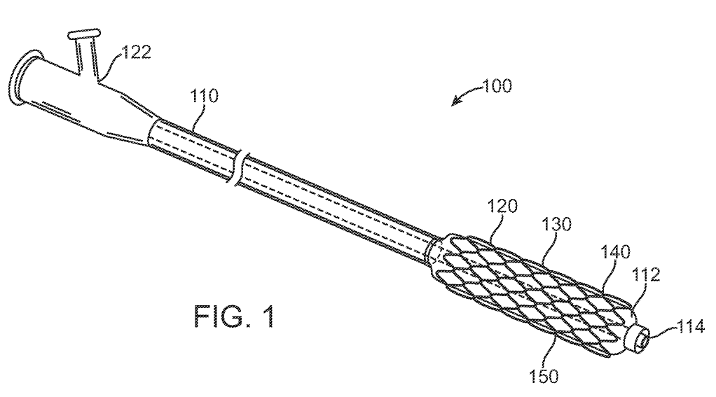

FIG. 1 illustrates one embodiment of a system 100 for treating a vascular condition. System 100 comprises nanoporous stent (stent) 120 coupled to catheter 110. Catheter 110 includes a balloon 112 that expands and deploys stent 120 within a vessel of the body. After positioning stent 120 within the vessel, balloon 112 is inflated by pressurizing a fluid such as a contrast fluid or saline solution that fills a lumen inside catheter 110 and balloon 112. Stent 120 is expanded until a desired diameter is reached; then the contrast fluid is depressurized or pumped out, separating balloon 112 from stent 120 and leaving stent 120 deployed in the vessel of the body. Alternately, catheter 110 may include a sheath that retracts to allow expansion of a self-expanding embodiment of stent 120.

Catheter 110 may comprise an elongated tubular member having a substantially circular cross-section and inside and outside walls that are substantially smooth. Catheter 110 may be secured at its proximal end to a suitable Luer fitting 122 and may include a distal rounded end to reduce harmful contact with a vessel. Catheter 110 may be manufactured substantially from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax®, Vestamid®, Tecoflex®, Halar®, Hyflon®, Pellathane®, combinations thereof, and the like. Catheter 110 may include lumen 114 formed therethrough allowing it to be advanced over a pre-positioned guidewire.

Balloon 112, shown in a collapsed state, may be any variety of balloon capable of expanding stent 120. Balloon 112 may be manufactured from any sufficiently elastic material such as polyethylene, polyethylene terephthalate (PET), nylon, or the like.

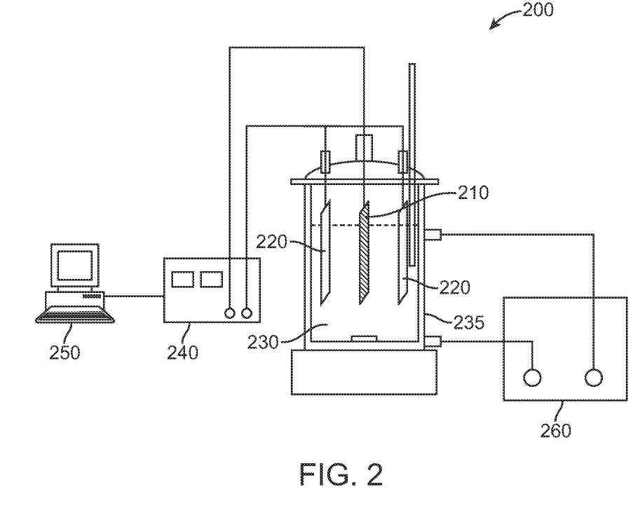

Stent 120 includes stent framework 130 comprising a plurality of stent struts and forming interior and exterior surfaces of the stent. In one embodiment, stent 120 comprises a plurality of stent segments 122 placed end-to end. Stent framework 130 is formed by shaping a metallic wire or filament, or by laser cutting the stent from a metallic sheet, or any other appropriate method. Stent 120 includes a nanoporous surface 150, described in more detail below with reference to FIGS. 2 to 4.

Stent 120 may also include a drug coating 140 disposed on and/or within nanoporous surface 150 of stent framework 130. In one embodiment, drug coating 140 includes at least one therapeutic agent or drug. Throughout, the terms “therapeutic agent” and “drug” are used interchangeably and refer to any agent that is a “biologically or pharmacologically active substance” whether synthetic or natural, that has a pharmacological, chemical, or biological effect on the body or a portion thereof. A therapeutic agent is capable of producing a beneficial effect against one or more conditions including coronary restenosis, cardiovascular restenosis, angiographic restenosis, arteriosclerosis, hyperplasia, and other diseases or conditions. The therapeutic agent may be, for example, anticoagulants, antiinflammatories, fibrinolytics, antiproliferatives, antibiotics, therapeutic proteins, recombinant DNA products, bioactive agents, diagnostic agents, radioactive isotopes, and radiopaque substances. In one embodiment, the biologically or pharmacologically active substance may be suspended in a polymer matrix or carrier. In one embodiment, the polymer matrix or carrier is biodegradable or bioresorbable such that it is absorbed in the body. The polymer matrix may comprise biodegradable polymers such as polylactic acid (PLA), polyglycolic acid, and their copolymers, polyethylene oxide (PEO), caproic acid, polyethylene glycol, polyanhydrides, polyacetates, polycaprolactones, poly(orthoesters), polyamides, polyurethanes and other suitable polymers.

Drug coating 140 containing the at least one therapeutic agent may additionally contain excipients including solvents or other solubilizers, stabilizers, suspending agents, antioxidants, and preservatives, as needed to deliver an effective dose of the therapeutic agent to the treatment site. The therapeutic agent coating 250 may be applied to the nanoporous surface 150 by any means known in the art such as, for example, by spraying, dipping, and brushing. In one embodiment, the coating is applied as a liquid by brushing or spraying, and then dried to remove solvent using air, vacuum, or heat, and any other effective means of causing the formulation to adhere to the stent framework. If needed, drug coating 140 is cured by exposure to ultraviolet light, heat, gamma irradiation or any other appropriate means.

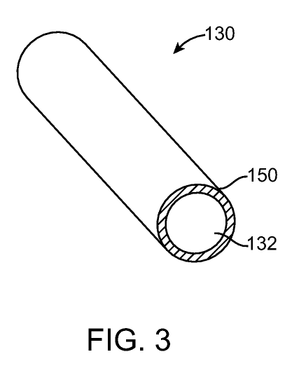

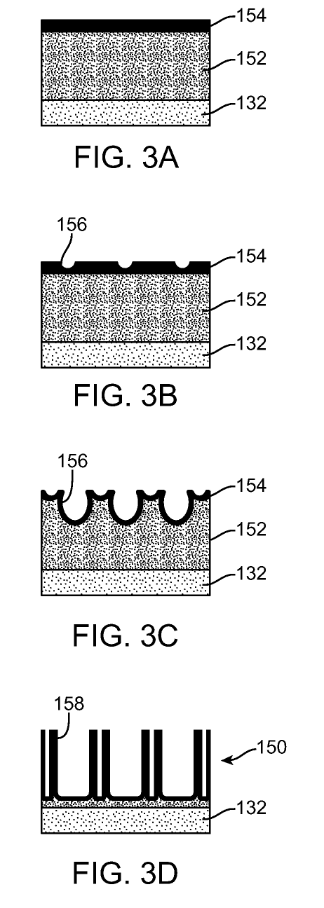

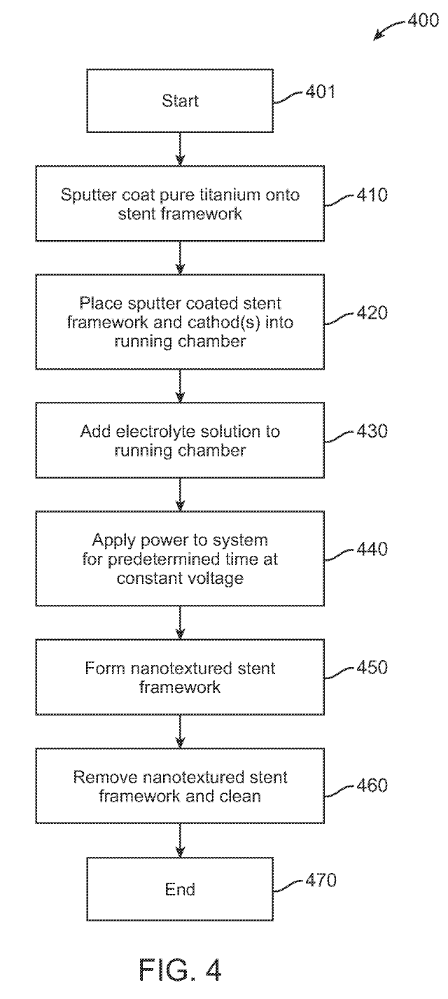

FIGS. 2 to 4 illustrate a system and method for forming a nanoporous surface on stent framework 130, in accordance with the present invention. FIG. 2 is a schematic illustration of one embodiment of a system for forming a nanoporous surface on stent framework 130. FIG. 3 is a cross section of a portion of a stent strut 134 comprising stent framework 130. FIGS. 3A to 3D are a series of illustrations that show the formation of a nanoporous layer 150 on stent framework 130. FIG. 4 is a flow chart of one embodiment of a method for forming a nanoporous surface on stent framework 130.

Stent framework 130 includes a base or core of metallic material 132 and a titanium oxide nanoporous layer 150 disposed on an outer surface of base 132 as shown in FIG. 3. Stent base 132 comprises a biocompatible metal or metal alloy. In one embodiment of the invention, the stent base 132 comprises one or more of a variety of biocompatible metals such as stainless steel, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys. The metal composition of stent base 132 gives the stent framework the mechanical strength to support the lumen wall of the vessel and sufficient longitudinal flexibility so that it can be transported through the cardiovascular system.

Nanoporous layer 150 comprises a layer of biocompatible titanium. An initial step in the manufacture of stent 120 includes depositing a uniform layer of pure titanium onto an outer surface of a bare metal stent or stent core such as core 132 using a sputter coating process. The pure titanium is sputter coated onto core 132 to a thickness of about 1 to 20 micrometers. Preferably, the titanium sputter coat is from 5 to 10 μm thick. The titanium is deposited to achieve a uniformly dense and uniformly thick layer of pure titanium on the outer surface of core 132. Once the desired thickness of pure titanium is deposited onto core 132, stent framework 130 is nanotextured using the nanotexturing system 200 illustrated in FIG. 2.

System 200 includes anode 210, cathode 220, solution 230, within solution (running) chamber 235, and power supply 240. In other embodiments, system 200 may also include computer 250 operably attached to power supply 240 for controlling the nanotexturing process. System 200 may also include cooling system 260 operably attached to running chamber 235.

Referring to FIG. 4, FIG. 4 is a flow chart illustrating one embodiment of a method 400 for manufacturing a medical device having a nanotextured coat of pure titanium. Method 400 begins at 401. Referring also to FIGS. 3A to 3D, a stent framework 130 is sputter coated to deposit a uniformly dense and thick layer of pure titanium 152 onto an outer surface of metallic core 132, (Block 410). The titanium may be deposited to a thickness of about 1 to 20 micrometers. In one embodiment, the titanium layer is deposited to a thickness of between 5 and 10 μm. In another embodiment, the titanium layer is deposited to a thickness of between 10 and 20 μm.

Next, at Block 420, the titanium coated stent framework 130 is placed in running chamber 235 of system 200 and connected to power source 240. Titanium coated stent framework 130 acts as an anode during the nanotexturing process. Platinum cathodes 220 are also placed in running chamber 235 and attached to power source 240. An electrolyte solution of 0.1 M H2SO4 and 0.1 M KF is added to running chamber 235 in amount that covers stent 130 (Block 430). At Block 440, the power source is turned on to provide a predetermined voltage to system 200 for a predetermined length of time. In one embodiment, the power supply is set at a constant voltage of between 1V and 30V. In one embodiment, the power is supplied for the predetermined length of time at a constant 5 volts. In one embodiment, the predetermined length of time for the application of the power supply is between one minute to two hours. In one embodiment, the predetermined length of time is between 30 and 60 minutes. In yet another embodiment, the predetermined length of time is between 3 and 5 minutes. The voltage and the running time are set to provide the desired nanotexture.

The nanotexture may range in size from nanopores to nanotubes. The size of the nanopores or nanotubes is determined by such factors as the thickness of the deposited titanium layer, the length of time the power is supplied to the stent framework and the voltage level supplied. At Block 450, the pure titanium layer is nanotextured.

FIG. 3A illustrates a portion of stent framework 130 as it would appear at an initial stage of the nanotexturing process. At the outset, a thin layer of titanium oxide 154 is formed on the surface of titanium layer 152. As the process continues, small pores 156 form in oxide layer 154 (FIG. 3B). Continued application of the electrical field causes the pores 156 to continue to grow in both depth and width within the pure titanium layer 152, as shown in FIG. 3C. A shown in FIG. 3D, in one embodiment, nanotubes 158 having a stable titanium oxide structure are formed in titanium layer 152. In one embodiment, nanotubes 1587 have a depth approximating the thickness of the titanium layer deposited on core 132. Those with ordinary skill in the art will appreciate that the nanotexturing process may be halted at any time to provide nanopores or nanotubes of a desired size. The desired size may depend on such factors as, the thickness of the titanium layer deposited in the core material, the overall size of the stent and the desired therapeutic dose of the therapeutic agent to be administered to the patient at the treatment site. At Block 460, the power is turned off and the nanotextured stent framework is removed and cleaned to remove any trace of the electrolyte solution. Method 400 ends at 470.

In use, the nanotextured stent is mounted at a distal end of a delivery catheter that is inserted into a patient's vascular system and delivered to the treatment site. The nanotexured stent may contain a therapeutic coating. The therapeutic coating may be applied to the stent as discussed above. At the treatment site, the stent is positioned across the lesion to be treated and is expanded into contact with the vessel wall. The catheter is then withdrawn from the body. In the physiological environment, the therapeutic agent(s) deposited within the plurality of nanopores are released from the stent surface.

While the invention has been described with reference to particular embodiments for treating a vascular condition, it will be understood by one skilled in the art that variations and modifications may be made in form and detail without departing from the spirit and scope of the invention.

1. A system for treating a vascular condition comprising:
a catheter;
a stent disposed on the catheter, the stent having a stent framework including a core, the core having an outer surface and a pure titanium layer deposited on the outer surface of the core; and
a nanotextured surface formed in the deposited pure titanium layer.

2. The system of claim 1 further comprising a therapeutic coating disposed in the nanotextured surface, the therapeutic coating including at least one therapeutic agent.

3. The system of claim 2 wherein the at least one therapeutic agent is selected from the group consisting of antirestenotic agents, anticoagulants, antiinflammatories, fibrinolytics, antiproliferatives, antibiotics, therapeutic proteins, recombinant DNA products, bioactive agents, diagnostic agents, radioactive isotopes, and radiopaque substances.

4. The system of claim 1 wherein the nanotextured surface comprises a plurality of nanopores.

5. The system of claim 1 wherein the nanotextured surface comprises a plurality of nanotubes.

6. The system of claim 1 wherein the core comprises a biocompatible metallic material.

7. The system of claim 6 wherein the biocompatible metallic material is selected from a group consisting of stainless steel, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys.

8. The system of claim 1 wherein the nanotextured surface comprises a plurality of stable titanium oxide structures.

9. A stent for treating a vascular condition, the stent comprising:
a stent framework including a core, the core having an outer surface;
a pure titanium layer deposited on the outer surface of the core; and
a nanotextured surface formed in the deposited pure titanium layer.

10. The stent of claim 9 further comprising a therapeutic coating disposed in the nanotextured surface, the therapeutic coating including at least one therapeutic agent.

11. The system of claim 1 wherein the at least one therapeutic agent is selected from the group consisting of antirestenotic agents, anticoagulants, antiinflammatories, fibrinolytics, antiproliferatives, antibiotics, therapeutic proteins, recombinant DNA products, bioactive agents, diagnostic agents, radioactive isotopes, and radiopaque substances.

12. The stent of claim 9 wherein the nanotextured surface comprises a plurality of nanopores.

13. The stent of claim 9 wherein the nanotextured surface comprises a plurality of nanotubes.

14. The stent of claim 9 wherein the core comprises a biocompatible metallic material.

15. The stent of claim 14 wherein the biocompatible metallic material is selected from a group consisting of stainless steel, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys.

16. The stent of claim 9 wherein the nanotextured surface comprises a plurality of stable titanium oxide structures.

17. A method of manufacturing a stent for treating a vascular condition, the method comprising:
depositing a uniformly dense and uniformly thick layer of pure titanium onto an outer surface of a stent framework core;
placing stent framework into a running chamber having a cathode and electrolyte solution;
connecting stent framework and cathode to a power source operably connected to the running chamber;
applying a power source to the attached stent framework and cathode for a predetermined length of time and at a predetermined voltage and
forming a nanotextured surface within the deposited titanium layer.

18. The method of claim 17 further comprising:
applying a therapeutic coating to the nanotextured surface, the therapeutic coating including at least one therapeutic agent.

19. The method of claim 18 wherein the at least one therapeutic agent is selected from the group consisting of antirestenotic agents, anticoagulants, antiinflammatories, fibrinolytics, antiproliferatives, antibiotics, therapeutic proteins, recombinant DNA products, bioactive agents, diagnostic agents, radioactive isotopes, and radiopaque substances.
